(19) United States
(12) Patent Application Publication
(10) Pub. No.: US 2015/0025284 A1
(43) Pub. Date: Jan. 22, 2015

(54) OXYGENATE REMOVAL FROM LIGHT HYDROCARBON PROCESSING

(71) Applicants: Clifford Michael Lowe, Moraga, CA (US); Robert Fletcher Cleverdon, Walnut Creek, CA (US)

(72) Inventors: Clifford Michael Lowe, Moraga, CA (US); Robert Fletcher Cleverdon, Walnut Creek, CA (US)

(73) Assignee: Chevron U.S.A. Inc., San Ramon, CA (US)

(21) Appl. No.: 13/944,476

(22) Filed: Jul. 17, 2013

(51) Int. Cl. C07C 7/12; C07C 2/56

(52) U.S. Cl. CPC C07C 7/12; C07C 2/56
USPC 585/324; 585/824

(57) ABSTRACT

Processes for eliminating water and oxygenates from a light hydrocarbon processing system, wherein oxygenates are removed from a light hydrocarbon stream by adsorption of the oxygenates on a primary oxygenate adsorption unit to provide a deoxygenated hydrocarbon stream, the primary oxygenate adsorption unit is regenerated via a first regenerant stream to provide an oxygenated first regenerant stream, the oxygenated first regenerant stream is deoxygenated via a secondary oxygenate adsorption unit, and the secondary oxygenate adsorption unit is regenerated via a second regenerant stream to provide an oxygenated second regenerant stream for permanent removal from the system.

### TECHNICAL FIELD

The present invention relates to the removal of oxygenates during light hydrocarbon processing.

### BACKGROUND

Various refinery and petrochemical processes involve reacting light olefins, to produce transportation fuels, plastics, and other commercial products, using catalyst systems that can be poisoned by contaminants in the olefin feed. Such contaminants may include water as well as various oxygenates, e.g., alcohols, ketones, carboxylic acids, and ethers.

Adsorbent materials for removing the water and oxygenates from the olefin feed become spent after use for a limited time period and must be regenerated for re-use to avoid excessive consumption and cost of the adsorbents. Spent adsorbent can be regenerated by desorbing the water and oxygenates into a stream of hot hydrocarbon vapor, e.g., isobutane. Such hydrocarbons may be valuable as feeds to various refinery processes. For example, isobutane is a valuable feed to ionic liquid alkylation. However, isobutane regenerant becomes contaminated with oxygenates and water during adsorbent regeneration. It is advantageous to remove the contaminants from the isobutane to prevent the accumulation of water and oxygenates, which could otherwise eventually break through the adsorbent beds and cause catalyst deactivation.

There is a need for processes that permanently remove water and oxygenates from light hydrocarbon processing systems in order to prevent contaminant accumulation in such systems, thereby protecting catalysts from deactivation by the contaminants.

### SUMMARY

In one embodiment there is provided a process for eliminating oxygenates from a light hydrocarbon processing system, the process comprising removing water and oxygenates from an olefin stream via a primary oxygenate adsorption unit to provide a deoxygenated olefin stream, wherein the primary oxygenate adsorption unit becomes spent; regenerating the spent primary oxygenate adsorption unit via a first regenerant stream to provide an oxygenated first regenerant stream comprising the water and the oxygenates; removing a portion of the water from the oxygenated first regenerant stream; removing residual water and the oxygenates from the oxygenated first regenerant stream via a secondary oxygenate adsorption unit to provide a deoxygenated first regenerant stream, wherein the secondary oxygenate adsorption unit becomes spent; and regenerating the spent secondary oxygenate adsorption unit via a second regenerant stream to provide an oxygenated second regenerant stream comprising the residual water and the oxygenates.

In another embodiment there is provided a process for eliminating oxygenates from a light hydrocarbon processing system, the process comprising adsorbing water and oxygenates from an olefin stream via a primary oxygenate adsorption unit to provide a deoxygenated olefin stream; when the primary oxygenate adsorption unit is spent, desorbing the water and the oxygenates from the primary oxygenate adsorption unit via a first regenerant stream to provide an oxygenated first regenerant stream comprising the water and the oxygenates; removing a portion of the water from the oxygenated first regenerant stream as condensate; via a secondary oxygenate adsorption unit, adsorbing residual water and the oxygenates from the oxygenated first regenerant stream; via a second regenerant stream, desorbing the water and the oxygenates from the secondary oxygenate adsorption unit to provide an oxygenated second regenerant stream comprising the residual water and the oxygenates; and permanently removing the oxygenated second regenerant stream from the light hydrocarbon processing system.

In a further embodiment there is provided a process for eliminating oxygenates from a light hydrocarbon processing system, the process comprising feeding an olefin stream to a primary oxygenate adsorption unit to provide a deoxygenated olefin stream; contacting the deoxygenated olefin stream and an isoparaffin stream with an ionic liquid catalyst in an ionic liquid alkylation zone under ionic liquid alkylation conditions; separating an alkylation hydrocarbon phase from an effluent of the ionic liquid alkylation zone; fractionating the alkylation hydrocarbon phase to provide an alkylate product; when the primary oxygenate adsorption unit becomes spent, regenerating the spent primary oxygenate adsorption unit via a first regenerant stream to provide an oxygenated first regenerant stream; removing oxygenates from the oxygenated first regenerant stream via a secondary oxygenate adsorption unit to provide a deoxygenated first regenerant stream; and when the secondary oxygenate adsorption unit becomes spent, regenerating the spent secondary oxygenate adsorption unit via a second regenerant stream to provide an oxygenated second regenerant stream.

As used herein, the terms “comprising” and “comprises” mean the inclusion of named elements or steps that are identified following those terms, but not necessarily excluding other unnamed elements or steps.

### BRIEF DESCRIPTION OF THE DRAWINGS

FIG. 1 schematically represents an oxygenate removal process for the elimination of oxygenates from hydrocarbon processing systems, according to an embodiment of the present invention;

FIG. 2 schematically represents the treatment of a primary oxygenate adsorption unit for the removal of residual olefins therefrom, according to another embodiment of the present invention;

FIG. 3 schematically represents the regeneration of a secondary oxygenate adsorption unit, according to another embodiment of the present invention; and

FIG. 4 schematically represents a system and process for ionic liquid catalyzed alkylation using a deoxygenated olefin stream, according to another embodiment of the present invention.

### DETAILED DESCRIPTION

Various refinery and petrochemical processes use light olefins, such as propene and butenes, as feeds to produce commercial products. An exemplary process is the alkylation of olefins with isobutane to produce high octane motor gasoline using ionic liquid catalysts. Refinery olefin streams, e.g., from a fluid catalytic cracking (FCC) unit, are typically contaminated with both water and oxygenates. It may be desirable or necessary to decrease the amount of water and/or oxygenates in olefin feeds for ionic liquid alkylation to very low levels before the olefin feed contacts the ionic liquid catalyst.

Adsorbent materials used for removing water and oxygenates from an olefin feed become spent after use for a limited time period. Spent adsorbent can be regenerated by desorbing the water and oxygenates into a regenerant stream, e.g., comprising hot hydrocarbon vapor. As disclosed herein, the oxygenates and water may be removed from the regenerant stream by passing the oxygenated regenerant stream over a secondary oxygenate adsorption unit. The oxygenates and water can be desorbed from the secondary oxygenate adsorption unit by a second regenerant stream for permanent removal of the oxygenates and water.

The term “deoxygenated” may be used herein to refer to a regenerant stream or hydrocarbon stream from which one or more oxygenates may have been adsorbed or otherwise removed, such that the hydrocarbon feed stream or regenerant stream may be depleted in the one or more oxygenates; a deoxygenated stream may be similarly depleted in water.

The term “oxygenated” may be used herein to refer to a regenerant stream into which one or more oxygenates may have been desorbed, such that the regenerant stream may be enriched in the one or more oxygenates; an oxygenated regenerant stream may be similarly enriched in water.

Applicants have found that oxygenate and water may be effectively eliminated from olefin streams to provide deoxygenated olefin streams. Such olefin streams may be suitable for light hydrocarbon processing, including ionic liquid catalyzed alkylation.

Oxygenate Removal for Light Hydrocarbon Processing

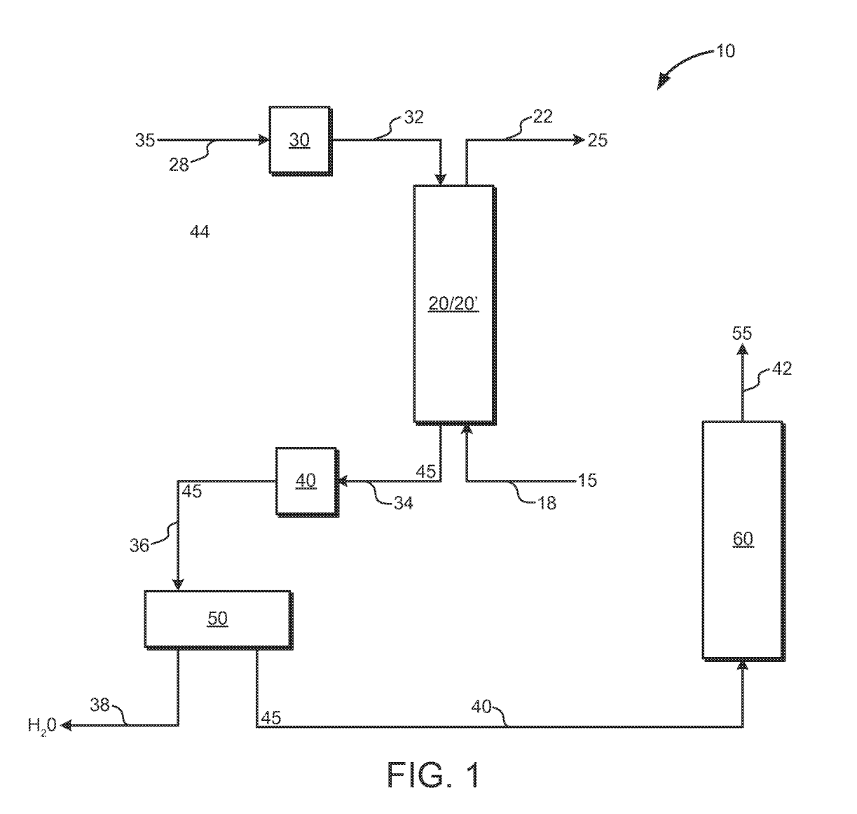

FIG. 1 schematically represents an oxygenate removal process for the elimination of oxygenates from light hydrocarbon processing systems, according to an embodiment of the present invention. System 10 may comprise a primary oxygenate adsorption unit 20/20′ that can be operated in an adsorption mode or a regeneration mode, 20, 20′, respectively. In the adsorption mode, an olefin stream 15 may be fed to primary oxygenate adsorption unit 20 via line 18. As an example, olefin stream 15 may comprise light olefins, such as C₃-C₅ olefins. Olefin stream 15 may be a raw or untreated olefin stream and may comprise water and/or oxygenate contaminants.

Primary oxygenate adsorption unit 20 may comprise an adsorbent for selectively adsorbing water and oxygenates from olefin stream 15. As a non-limiting example, the adsorbent of primary oxygenate adsorption unit 20 may comprise at least one of a molecular sieve and a metal oxide. Non-limiting examples of adsorbents for use in primary oxygenate adsorption unit 20 include a molecular sieve selected from the group consisting of s silicates, aluminosilicates, aluminophosphates, silicoaluminophosphates, and combinations thereof. In a sub-embodiment, an adsorbent for use in a secondary oxygenate adsorption unit 60 may comprise a zeolite, such as zeolite 13X. The adsorbent of primary oxygenate adsorption unit 20 may be disposed in at least one adsorbent bed (not shown).

Primary oxygenate adsorption unit 20/20′ may be operated in the adsorption mode or the regeneration mode. The regeneration mode may also be referred to herein as a desorption mode. FIG. 1 shows the operation of primary oxygenate adsorption unit 20/20′ in the adsorption mode and in the regeneration mode, it being understood that primary oxygenate adsorption unit 20/20′ may be operated alternately in the adsorption and regeneration modes.

During the adsorption mode of primary oxygenate adsorption unit 20, water and oxygenate contaminants may be adsorbed from olefin stream 15. In an embodiment, during the adsorption mode, more than one primary oxygenate adsorption unit 20 may be arranged in series for the adsorption of water and oxygenates from olefin stream 15. During the adsorption mode, primary oxygenate adsorption unit 20 may be maintained at a temperature typically in the range from 50 to 150° F. (10 to 65.56 degree Celsius), or from 70 to 130° F. (21.11 to 54.44 degree Celsius). The feed of olefin stream 15 to primary oxygenate adsorption unit 20 may be either upflow or downflow.

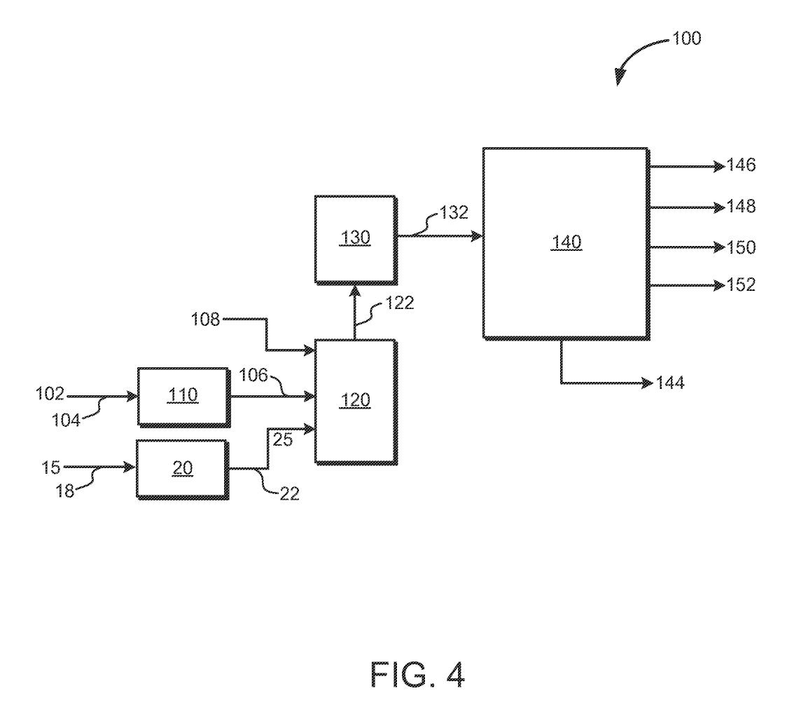

During the adsorption mode, a deoxygenated olefin stream 25 may be obtained from primary oxygenate adsorption unit 20. The expression “deoxygenated olefin stream” may be used herein to refer to an olefin stream that is depleted in oxygenates as compared with an untreated olefin stream. A deoxygenated olefin stream 25 (e.g., FIGS. 1 and 4) may also be depleted in water as compared with an untreated olefin stream, it being understood that water may be removed from an untreated olefin stream concurrently with oxygenate removal, e.g., by passage of the olefin stream through primary oxygenate adsorption unit 20.

In an embodiment, deoxygenated olefin stream 25 may have an oxygenate content of not more than 5 ppmw, not more than 2 ppmw, or not more than 1 ppmw. In an embodiment, deoxygenated olefin stream 25 may have a water content of not more than 5 ppmw, not more than 2 ppmw, or not more than 1 ppmw. Deoxygenated olefin stream 25 may be fed via line 22 to one or more downstream unit operations. In an embodiment, deoxygenated olefin stream 25 may be fed to an ionic liquid alkylation zone (see, for example, FIG. 4).

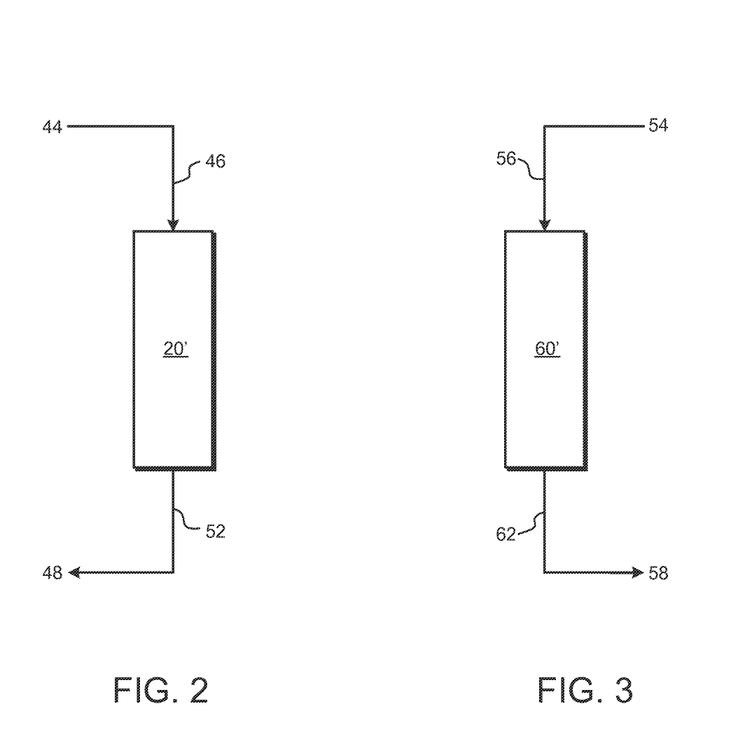

Although only one primary oxygenate adsorption unit 20/20′ is shown in FIG. 1, a plurality of such units may be used for treating an olefin stream. When a primary oxygenate adsorption unit 20 becomes spent, e.g., its capacity for the adsorption of water and/or oxygenates is exhausted, the feed of olefin stream 15 thereto may be terminated. Thereafter, the spent primary oxygenate adsorption unit 20′ may be regenerated by a first regenerant stream 35, as described hereinbelow, while a parallel primary oxygenate adsorption unit 20 may be put online to receive olefin stream 15. In an embodiment, prior to the regeneration of a spent primary oxygenate adsorption unit 20′, residual olefins may be recovered from the spent primary oxygenate adsorption unit 20′ (see, for example, FIG. 2).

FIG. 2 schematically represents the treatment of a spent primary oxygenate adsorption unit 20′ for the removal of residual olefins therefrom, according to another embodiment of the present invention. A primary oxygenate adsorption unit that is spent may be designated herein as spent primary oxygenate adsorption unit 20′. As described with reference to FIG. 1, supra, when primary oxygenate adsorption unit 20 is spent, the feed of olefin stream 15 thereto may be terminated, and the spent primary oxygenate adsorption unit 20′ may be taken offline for regeneration.

With further reference to FIG. 2, residual olefins 48 may be recovered (e.g., flushed) from spent primary oxygenate adsorption unit 20′ by feeding a flushing stream 44 to spent primary oxygenate adsorption unit 20′ via line 46. Flushing stream 44 may comprise a hydrocarbon stream, e.g., comprising isobutane. Flushing stream 44 may have a temperature typically not more than 150° F. (65.56 degree Celsius), or in the range from 50° F. (10 degree Celsius) to 150° F. (65.56 degree Celsius). In an embodiment, residual olefins 48 may be combined, via line 52, with olefin stream 15. Following the recovery of residual olefins 48, spent primary oxygenate adsorption unit 20′ may be regenerated, e.g., as described hereinbelow. In an embodiment, a step of recovering the residual olefins 48 from spent primary oxygenate adsorption unit 20′may be omitted.

With further reference to FIG. 1, for the regeneration of spent primary oxygenate adsorption unit 20′, a first regenerant stream 35 may be fed via line 28 to a heating unit 30 such that first regenerant stream 35 attains a temperature of at least 250° F. (121.1 degree Celsius), and typically the first regenerant stream 35 may attain a temperature in the range from 300 to 600° F. (148.9 to 315.6 degree Celsius). In an embodiment, heating unit 30 may comprise a heat exchanger.

A first regenerant stream 35 that has been heated may be fed via line 32 to spent primary oxygenate adsorption unit 20′. In an embodiment, the feed of the first regenerant stream 35 that has been heated to the spent primary oxygenate adsorption unit 20′ (regeneration mode) may be in a direction opposite to that of olefin stream 15 to primary oxygenate adsorption unit 20 (adsorption mode). In an embodiment, first regenerant stream 35 may comprise hydrocarbon vapor, e.g., comprising isobutane.

Water and oxygenates may be desorbed from the spent primary oxygenate adsorption unit 20′ by first regenerant stream 35 to provide an oxygenated first regenerant stream 45 comprising the water and oxygenates. The oxygenated first regenerant stream 45 may be fed via line 34 to a cooling unit 40 for cooling oxygenated first regenerant stream 45. In an embodiment, cooling unit 40 may comprise a heat exchanger.

In an embodiment, an oxygenated first regenerant stream 45 that has been cooled may be sent via line 36 to a decanter vessel 50 such that a portion of the desorbed water may be separated as condensate. The resultant free water, which may comprise some oxygenates, may be permanently removed via line 38, e.g. to a waste water treatment unit (not shown). In an embodiment, at least 90 wt % of the desorbed water, or at least 95 wt % of the desorbed water, in oxygenated first regenerant stream 45 may be condensed in decanter vessel 50 and removed therefrom as free water.

Decanter vessel 50 may include a baffle (not shown) to facilitate the separation of a liquid hydrocarbon phase from the condensed water. The liquid hydrocarbon phase of decanter vessel 50 may comprise oxygenated first regenerant stream 45. Oxygenated first regenerant stream 45 may comprise first regenerant stream 35 together with the major portion of desorbed oxygenates and some residual water.

Attention will now be directed, with reference to FIGS. 1 and 3, to the use of secondary oxygenate adsorption unit 60/60′ for the adsorption of oxygenates and water from oxygenated first regenerant stream 45, and the subsequent desorption and elimination of the oxygenates. Secondary oxygenate adsorption unit 60/60′ may be operated in an adsorption mode or a regeneration mode, 60, 60′, respectively. FIG. 1 shows the operation of secondary oxygenate adsorption unit 60 in the adsorption mode, while FIG. 3 shows the operation of a spent secondary oxygenate adsorption unit 60′ in the regeneration (or desorption) mode.

With further reference to FIG. 1, oxygenated first regenerant stream 45 may be fed via line 40 to secondary oxygenate adsorption unit 60 for the adsorption of the oxygenates and residual water from oxygenated first regenerant stream 45 to provide a deoxygenated first regenerant stream 55. In an embodiment, deoxygenated first regenerant stream 55 may have an oxygenate content of not more than 50 ppmw, not more than 10 ppmw, not more than 5 ppmw, or not more than 1 ppmw. In an embodiment, deoxygenated first regenerant stream 55 may comprise isobutane.

In an embodiment, deoxygenated first regenerant stream 55 may have a water content of not more than 50 ppmw, not more than 10 ppmw, not more than 5 ppmw, or not more than 1 ppmw. In an embodiment, deoxygenated first regenerant stream 55 may be sent via line 42 to one or more downstream unit operations, e.g., to an ionic liquid alkylation zone (see, for example, FIG. 4). In another embodiment, deoxygenated first regenerant stream 55 may be recycled, e.g., by combination with olefin stream 15, to primary oxygenate adsorption unit 20.

Secondary oxygenate adsorption unit 60 may comprise at least one adsorbent. As a non-limiting example, an adsorbent of secondary oxygenate adsorption unit 60 may comprise at least one of a molecular sieve and a metal oxide. Non-limiting examples of adsorbents that may be used in secondary oxygenate adsorption unit 60 include a molecular sieve selected from the group consisting of silicates, aluminosilicates, aluminophosphates, silicoaluminophosphates, and combinations thereof. In a sub-embodiment, an adsorbent for use in secondary oxygenate adsorption unit 60 may comprise a zeolite, such as zeolite 13X. The adsorbent of secondary oxygenate adsorption unit 60 may be disposed in at least one adsorbent bed (not shown).

Although only one secondary oxygenate adsorption unit 60 is shown in FIG. 1, a plurality of such units may be used for the treatment of oxygenated first regenerant stream 45. When a secondary oxygenate adsorption unit 60 becomes spent, e.g., its capacity for the adsorption of water and/or oxygenates is exhausted, the feed of oxygenated first regenerant stream 45 may be terminated and the spent secondary oxygenate adsorption unit 60′ may be taken offline for regeneration, while a parallel secondary oxygenate adsorption unit 60 may be put online in the adsorption mode to receive oxygenated first regenerant stream 45. The spent secondary oxygenate adsorption unit 60′ may be regenerated by a second regenerant stream 54, as described hereinbelow (see, for example, FIG. 3).

FIG. 3 schematically represents the regeneration of a secondary oxygenate adsorption unit, according to another embodiment of the present invention. In order to avoid the recycling and accumulation of oxygenates and water in the olefin treatment equipment, a spent secondary oxygenate adsorption unit 60′ may be regenerated using a second regenerant stream 54. Second regenerant stream 54 may be fed to spent secondary oxygenate adsorption unit 60′ via line 56 such that oxygenates and/or water are desorbed from the spent secondary oxygenate adsorption unit 60′, whereby spent secondary oxygenate adsorption unit 60′ may be regenerated.

In an embodiment, second regenerant stream 54 may be heated to a temperature of at least 250° F. (121.1 degree Celsius), and typically to a temperature in the range from 300 to 600° F. (148.9 to 315.6 degree Celsius), prior to its introduction into spent secondary oxygenate adsorption unit 60′. The effluent from spent secondary oxygenate adsorption unit 60′, which may comprise second regenerant stream 54 together with desorbed oxygenates and water, may be referred to herein as an oxygenated second regenerant stream 58.

Oxygenated second regenerant stream 58, including the desorbed oxygenates and water therein, may be permanently removed, via line 62, from the olefin treating equipment and from any downstream unit operations that may require the use of treated olefin feeds. In an embodiment, oxygenated second regenerant stream 58 may be combusted. In an embodiment, oxygenated second regenerant stream 58 may be combined with a combustible gas prior to combustion. In an embodiment, second regenerant stream 54 may comprise fuel gas, and the oxygenated second regenerant stream 58 may be sent to a refinery fuel gas header (not shown) for combustion.

In an embodiment, there is provided herein a process for eliminating oxygenates from a light hydrocarbon processing system. Such process may comprise removing water and oxygenates from an olefin stream 15 via a primary oxygenate adsorption unit 20 to provide a deoxygenated olefin stream 25, wherein the water and oxygenates may be adsorbed by primary oxygenate adsorption unit 20. As a result, primary oxygenate adsorption unit 20 will eventually become spent. Spent primary oxygenate adsorption unit 20′ may be regenerated via a first regenerant stream 35 to provide an oxygenated first regenerant stream 45. The oxygenated first regenerant stream 45 may comprise water and oxygenates desorbed from spent primary oxygenate adsorption unit 20′ by first regenerant stream 35. First regenerant stream 35 may be at a temperature of at least 250° F. (121.1 degree Celsius), or at a temperature in the range from 300 to 600° F. (148.9 to 315.6 degree Celsius). In an embodiment, first regenerant stream 35 may comprise isobutane.

A portion of the water may be removed from the oxygenated first regenerant stream 45. In an embodiment, such water may be removed as condensate by cooling the oxygenated first regenerant stream 45, and thereafter feeding the oxygenated first regenerant stream 45 to a decanter vessel 50. The condensed free water may be eliminated from the system, e.g., by sending the condensed water to a waste water treatment unit. In one embodiment, the removing a portion of the water from the oxygenated first regenerant stream 45 comprises: cooling the oxygenated first regenerant stream 45, and condensing the portion of the water from the oxygenated first regenerant stream 45.

Water that remains in oxygenated first regenerant stream 45 after water removal as condensate by decanter vessel 50 may be referred to as “residual water.” A process for eliminating oxygenates from a light hydrocarbon processing system may further comprise removing the residual water and the oxygenates from the oxygenated first regenerant stream 45 via a secondary oxygenate adsorption unit 60 to provide a dried, deoxygenated first regenerant stream 55 (see, for example, FIG. 1). As a result of the latter step, the secondary oxygenate adsorption unit 60 may become spent.

The spent secondary oxygenate adsorption unit 60′ may be regenerated via a second regenerant stream 54 to provide an oxygenated second regenerant stream 58 (see, e.g., FIG. 3). Oxygenated second regenerant stream 58 may comprise residual water desorbed from spent secondary oxygenate adsorption unit 60′. Oxygenated second regenerant stream 58 may further comprise oxygenates desorbed from spent secondary oxygenate adsorption unit 60′. Oxygenated second regenerant stream 58 may be permanently removed from the light hydrocarbon processing system.

In an embodiment, the second regenerant stream 54 may comprise fuel gas, and the oxygenated second regenerant stream 58 (effluent) from the spent secondary oxygenate adsorption unit 60′may be fed to a refinery fuel gas header for combustion. Fuel gas used as second regenerant stream 54 may be at a temperature of at least 250° F. (121.1 degree Celsius), or in the range from 300 to 600° F. (148.9 to 315.6 degree Celsius).

In an embodiment, residual olefins may be recovered from the spent primary oxygenate adsorption unit 20′ prior to regeneration thereof. In a sub-embodiment, recovery of the residual olefins from the spent primary oxygenate adsorption unit 20′ may comprise flushing the residual olefins from the spent primary oxygenate adsorption unit 20′ with isobutane. Such isobutane for flushing the residual olefins from the spent primary oxygenate adsorption unit may have a temperature of not more than 150° F. (65.56 degree Celsius), or from 50 to 150° F. (10 to 65.56 degree Celsius). In an embodiment, the residual (flushed) olefins may be combined with olefin stream 15, or may be fed to a FCC Gas Recovery Unit.

An olefin stream 15 that is fed to primary oxygenate adsorption unit 20 may be raw or untreated. In an embodiment, olefin stream 15 may be from a FCC unit. Olefin stream 15 may be contaminated with both water and various oxygenates. Olefin stream 15 may be saturated with water vapor. In an embodiment, olefin stream 15 may have a water content of at least 300 ppmw, or in the range from 300 to 500 ppmw. In contrast, the deoxygenated olefin stream 25 provided by primary oxygenate adsorption unit 20 may have a water content of not more than 5 ppmw, not more than 2 ppmw, or not more than 1 ppmw.

In an embodiment, deoxygenated olefin stream 25 may be fed, together with an isoparaffin stream, to an ionic liquid alkylation zone for contact with an ionic liquid catalyst under ionic liquid alkylation conditions to provide an ionic liquid alkylate (see, for example, FIG. 4).

In another embodiment, there is provided herein a process for eliminating oxygenates from a light hydrocarbon processing system. Such process may comprise adsorbing water and oxygenates from an olefin stream 15 via a primary oxygenate adsorption unit 20. Although only one primary oxygenate adsorption unit 20/20′ is shown in FIG. 1, a plurality of such units may be used for olefin feed treatment, and each such unit may be operated alternately in an adsorption mode and a desorption (regeneration) mode. When the capacity of a primary oxygenate adsorption unit 20 for the adsorption of water and oxygenates is exhausted, it may be referred to as a spent primary oxygenate adsorption unit 20′.

Water and oxygenates may be desorbed from a spent primary oxygenate adsorption unit 20′ via a first regenerant stream 35 to provide an oxygenated first regenerant stream 45. Such oxygenated first regenerant stream 45 may comprise the desorbed water and oxygenates from spent primary oxygenate adsorption unit 20′.

A portion of the water may be removed from oxygenated first regenerant stream 45 as condensate. In an embodiment, the removal of the condensate (free water) from oxygenated first regenerant stream 45 may comprise cooling the oxygenated first regenerant stream 45. In an embodiment, oxygenated first regenerant stream 45 may be cooled to a temperature in the range from 60 to 130° F. (15.56 to 54.44 degree Celsius), or from 70 to 120° F. (21.11 to 48.89 degree Celsius).

An oxygenated first regenerant stream 45 that has been cooled may be fed to a decanter vessel 50 for the separation of the condensed water from a liquid hydrocarbon phase. Decanter vessel 50 may comprise a vertical baffle (not shown) to promote separation of the aqueous and hydrocarbon phases. The free water separated by decanter vessel 50 may be permanently removed from the system, e.g., by sending it to a waste water treatment unit.

The oxygenated first regenerant stream 45 (hydrocarbon phase) from decanter vessel 50 may comprise the hydrocarbons of first regenerant stream 35 together with some residual water and the major portion of the oxygenates desorbed from primary oxygenate adsorption unit 20. Oxygenated first regenerant stream 45 may be fed to a secondary oxygenate adsorption unit 60 for the adsorption of the oxygenates and the residual water. As a result of such adsorption of the residual water and the oxygenates from oxygenated first regenerant stream 45, secondary oxygenate adsorption unit 60 eventually becomes spent.

Although only one secondary oxygenate adsorption unit 60 is shown in FIG. 1, a plurality of such units may be used for the treatment of oxygenated first regenerant stream 45. For example, when the adsorption capacity of a secondary oxygenate adsorption unit 60 is spent, the spent secondary oxygenate adsorption unit 60′ may be taken offline, while a parallel secondary oxygenate adsorption unit 60 may be put online to receive oxygenated first regenerant stream 45. Spent secondary oxygenate adsorption unit 60′ may then be regenerated by desorbing the water and the oxygenates via a second regenerant stream 54 (see, e.g., FIG. 3) to provide an oxygenated second regenerant stream 58 comprising the desorbed water and oxygenates. Oxygenated second regenerant stream 58 may then be permanently removed from the system.

In an embodiment, the permanent removal of oxygenated second regenerant stream 58 may comprise combusting of the oxygenated second regenerant stream 58. In an embodiment, the permanent removal of oxygenated second regenerant stream 58 may comprise combining the oxygenated second regenerant stream 58 with a combustible gas. In an embodiment, second regenerant stream 54 may comprise fuel gas, and oxygenated second regenerant stream 58 may be fed to a refinery fuel gas header for combustion.

In an embodiment, a deoxygenated olefin stream 25 and at least one isoparaffin may be contacted with an ionic liquid catalyst in an ionic liquid alkylation zone under ionic liquid alkylation conditions. An alkylation hydrocarbon phase may be separated from an effluent of the ionic liquid alkylation zone, and the alkylation hydrocarbon phase may be fractionated to provide an alkylate product (see, for example, FIG. 4).

FIG. 4 schematically represents a system and process for ionic liquid catalyzed alkylation, according to another embodiment of the present invention. Such system and process may use a dry, deoxygenated olefin stream as a feed for the ionic liquid alkylation reaction. Ionic liquid alkylation system 100 (FIG. 4) provides a non-limiting example of a light hydrocarbon processing system to which oxygenate removal processes of the present invention may be applied.

A process for the preparation of ionic liquid alkylate will now be described with reference to FIG. 4. Olefin feed 15 may be fed via line 18 to primary oxygenate adsorption unit 20 to provide a deoxygenated olefin stream 25, e.g., essentially as described with reference to FIG. 1, supra. At the same time, an isoparaffin stream 102 may be fed via line 104 to an isoparaffin dryer 110 to provide a dried isoparaffin stream. The deoxygenated olefin stream 25 and the dried isoparaffin stream may be fed, via lines 22 and 106, respectively, to an ionic liquid alkylation zone 120 together with an ionic liquid catalyst 108.

In an embodiment, isoparaffin stream 102 may comprise deoxygenated first regenerant stream 55 from secondary oxygenate adsorption unit 60 (see, e.g., FIG. 1). In an embodiment, deoxygenated first regenerant stream 55 may be fed directly to ionic liquid alkylation zone 120, e.g., isoparaffin dryer 110 may be circumvented. In an embodiment, deoxygenated first regenerant stream 55 may comprise isobutane. In an embodiment, deoxygenated first regenerant stream 55 may comprise not more than 50 ppmw of water, or not more than 10 ppmw, or not more than 5 ppmw, or not more than 1 ppmw of water. In an embodiment, deoxygenated first regenerant stream 55 may comprise not more than 50 ppmw of oxygenates, or not more than 10 ppmw, or not more than 5 ppmw, or not more than 1 ppmw of oxygenates.

In ionic liquid alkylation zone 120, at least one isoparaffin and at least one olefin may be contacted with ionic liquid catalyst 108 under ionic liquid alkylation conditions. Anhydrous HCl co-catalyst or an organic chloride catalyst promoter (neither of which are shown) may be combined with ionic liquid catalyst 108 in ionic liquid alkylation zone 120 to attain the desired level of catalytic activity and selectivity for the alkylation reaction.

Ionic liquid alkylation conditions, feedstocks, and ionic liquid catalysts that may be suitable for performing ionic liquid alkylation reactions in ionic liquid alkylation system 100 are described, for example, hereinbelow.

The effluent from ionic liquid alkylation zone 120 may be fed via line 122 to an ionic liquid/hydrocarbon (IL/HC) separator 130 for the separation of a hydrocarbon phase from the effluent. Non-limiting examples of separation processes that can be used for separating the hydrocarbon phase from the effluent include coalescence, phase separation, extraction, membrane separation, and partial condensation. IL/HC separator 130 may comprise, for example, one or more of the following: a settler, a coalescer, a centrifuge, a distillation column, a condenser, and a filter.

The hydrocarbon phase from IL/HC separator 130 may be fed via a line 132 to an ionic liquid alkylate separation system 140. The hydrocarbon phase from IL/HC separator 130 may be referred to herein as an alkylation hydrocarbon phase. Ionic liquid alkylate separation system 140 may comprise at least one distillation unit (not shown). The alkylation hydrocarbon phase from IL/HC separator 130 may be fractionated via ionic liquid alkylate separation system 140 to provide an alkylate product 144, as well as HCl 146, a propane fraction 148, an n-butane fraction 150, and an isobutane fraction 152.

The instant specification also provides a process for eliminating oxygenates from a light hydrocarbon processing system. With further reference to FIGS. 1, 3, and 4, oxygenates may be effectively removed from an olefin stream 15 by feeding the olefin stream 15 to primary oxygenate adsorption unit 20 in the adsorption mode to provide a deoxygenated olefin stream 25. Primary oxygenate adsorption unit 20 may also remove water from olefin stream 15 concomitantly with the removal of oxygenates. In an embodiment, deoxygenated olefin stream 25 may have a water content of not more than 5 ppmw, not more than 2 ppmw, or not more than 1 ppmw. In an embodiment, deoxygenated olefin stream 25 may have an oxygenate content of not more than 5 ppmw, not more than 2 ppmw, or not more than 1 ppmw.

The deoxygenated olefin stream 25 and an isoparaffin stream may be contacted with an ionic liquid catalyst in an ionic liquid alkylation zone 120 under ionic liquid alkylation conditions. In an embodiment, the isoparaffin stream may comprise deoxygenated first regenerant stream 55 (see, e.g., FIG. 1). An alkylation hydrocarbon phase may be separated from an effluent of the ionic liquid alkylation zone 120, e.g., using an IL/HC separator 130. Thereafter, the alkylation hydrocarbon phase from IL/HC separator 130 may be fractionated, e.g., via an ionic liquid alkylate separation system 140, to provide, inter alia, an alkylate product.

When a primary oxygenate adsorption unit 20 becomes spent, the feed of olefin stream 15 to the spent primary oxygenate adsorption unit 20′ may be terminated, preparatory to operation of the spent primary oxygenate adsorption unit 20′ in the regeneration mode. Spent primary oxygenate adsorption unit 20′ may be regenerated via a first regenerant stream 35 to provide an oxygenated first regenerant stream 45. Oxygenated first regenerant stream 45 may comprise desorbed oxygenates and water. In an embodiment, a portion of the water may be removed from oxygenated first regenerant stream 45 as condensate water (i.e., free water). Thereafter, the oxygenates may be removed from oxygenated first regenerant stream 45 via a secondary oxygenate adsorption unit 60 to provide a deoxygenated first regenerant stream 55. Secondary oxygenate adsorption unit 20 may also remove residual water from oxygenated first regenerant stream 45 concomitantly with the removal of oxygenates therefrom.

As a result of the adsorption of oxygenates from oxygenated first regenerant stream 45 via secondary oxygenate adsorption unit 60, secondary oxygenate adsorption unit 60 may become spent. With reference to FIG. 3, spent secondary oxygenate adsorption unit 60′ may be regenerated via a second regenerant stream 54 to provide an oxygenated second regenerant stream 58. Thereafter, oxygenated second regenerant stream 58 may be eliminated from the system. In an embodiment, oxygenated second regenerant stream 58 may be eliminated by combustion in admixture with refinery fuel gas.

Ionic Liquid Catalyzed Alkylation

Ionic liquid catalysts may be useful for a range of hydrocarbon conversion reactions, including alkylation reactions for the production of alkylate, e.g., comprising gasoline blending components, and the like. In an embodiment, feedstocks for ionic liquid catalyzed alkylation may comprise various olefin- and isoparaffin containing hydrocarbon streams in or from one or more of the following: a petroleum refinery, a gas-to-liquid conversion plant, a coal-to-liquid conversion plant, a naphtha cracker, a middle distillate cracker, and a wax cracker, and the like.

Examples of olefin containing streams include FCC off-gas, coker gas, olefin metathesis unit off-gas, polyolefin gasoline unit off-gas, methanol to olefin unit off-gas, FCC light naphtha, coker light naphtha, Fischer-Tropsch unit products, and cracked naphtha. Some olefin containing streams may contain two or more olefins selected from ethylene, propylene, butylenes, pentenes, and up to C₁₀ olefins. Such olefin containing streams are further described, for example, in U.S. Pat. No. 7,572,943, the disclosure of which is incorporated by reference herein in its entirety.

Examples of isoparaffin containing streams include, but are not limited to, FCC naphtha, hydrocracker naphtha, coker naphtha, Fisher-Tropsch unit products, and cracked naphtha. Such streams may comprise at least one C₄-C₁₀ isoparaffin. In an embodiment, such streams may comprise a mixture of two or more isoparaffins. In a sub-embodiment, an isoparaffin feed to the alkylation reactor during an ionic liquid catalyzed alkylation process may comprise isobutane.

Various ionic liquids may be used as catalysts for alkylation reactions involving olefins. Ionic liquids are generally organic salts with melting points below 100° C. (212 degree Fahrenheit) and often below room temperature. The use of chloroaluminate ionic liquids as alkylation catalysts in petroleum refining has been described, for example, in commonly assigned U.S. Pat. Nos. 7,531,707, 7,569,740, and 7,732,654, the disclosure of each of which is incorporated by reference herein in its entirety. Exemplary ionic liquids for use as catalysts in ionic liquid catalyzed alkylation reactions may comprise at least one compound of the general formulas A and B:

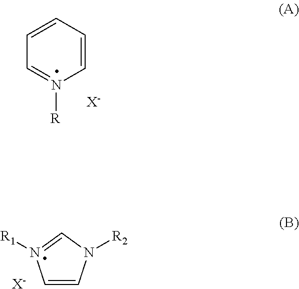

wherein R is H, methyl, ethyl, propyl, butyl, pentyl or hexyl, each of R₁ and R ₂ is H, methyl, ethyl, propyl, butyl, pentyl or hexyl, wherein R₁ and R₂ may or may not be the same, and X is a chloroaluminate.

Non-limiting examples of chloroaluminate ionic liquid catalysts that may be used in alkylation processes according to embodiments of the instant invention include those comprising 1-butyl-4-methyl-pyridinium chloroaluminate, 1-butyl-3-methyl-imidazolium chloroaluminate, 1-H-pyridinium chloroaluminate, N-butylpyridinium chloroaluminate, and mixtures thereof.

Exemplary reaction conditions for ionic liquid catalyzed alkylation are as follows. The ionic liquid alkylation reaction temperature may be generally in the range from −40° C. (−40 degree Fahrenheit) to +250° C. (482 degree Fahrenheit), typically from −20° C. to +100° C. (−4° F. to +212° F.), and often from +4° C. to +60° C. (+39.2° F. to +140° F.). The ionic liquid alkylation reactor pressure may be in the range from atmospheric pressure to 8000 kPa. Typically, the pressure in the ionic liquid alkylation zone 120 is sufficient to keep the reactants in the liquid phase.

Residence time of reactants in ionic liquid alkylation zone 120 may generally be in the range from a few seconds to hours, and usually from 0.5 min to 60 min. A feed stream introduced into ionic liquid alkylation zone 120 may have an isoparaffin:olefin molar ratio generally in the range from 1 to 100, more typically from 2 to 50, and often from 2 to 20.

The volume of ionic liquid catalyst in ionic liquid alkylation zone 120 may be generally in the range from 1 to 70 vol %, and usually from 4 to 50 vol %. The ionic liquid alkylation conditions may be adjusted to optimize process performance for a particular process or targeted product(s).

Numerous variations on the present invention are possible in light of the teachings described herein. It is therefore understood that within the scope of the following claims, the invention may be practiced otherwise than as specifically described or exemplified herein.

For the purposes of this specification and appended claims, unless otherwise indicated, all numbers expressing quantities, percentages or proportions, and other numerical values used in the specification and claims, are to be understood as being modified in all instances by the term “about.” Furthermore, all ranges disclosed herein are inclusive of the endpoints and are independently combinable. Whenever a numerical range with a lower limit and an upper limit are disclosed, any number falling within the range is also specifically disclosed.

Any term, abbreviation or shorthand not defined is understood to have the ordinary meaning used by a person skilled in the art at the time the application is filed. The singular forms “a,” “an,” and “the,” include plural references unless expressly and unequivocally limited to one instance.

All of the publications, patents and patent applications cited in this application are herein incorporated by reference in their entirety to the same extent as if the disclosure of each individual publication, patent application or patent was specifically and individually indicated to be incorporated by reference in its entirety.

This written description uses examples to disclose the invention, including the best mode, and also to enable any person skilled in the art to make and use the invention. Many modifications of the exemplary embodiments of the invention disclosed above will readily occur to those skilled in the art. Accordingly, the invention is to be construed as including all structure and methods that fall within the scope of the appended claims. Unless otherwise specified, the recitation of a genus of elements, materials or other components, from which an individual component or mixture of components can be selected, is intended to include all possible sub-generic combinations of the listed components and mixtures thereof.

The invention illustratively disclosed herein suitably may be practiced in the absence of any element which is not specifically disclosed herein.

What is claimed is:

1. A process for eliminating oxygenates from a light hydrocarbon processing system, the process comprising:
a) removing water and the oxygenates from an olefin stream via a primary oxygenate adsorption unit to provide a deoxygenated olefin stream, wherein the primary oxygenate adsorption unit becomes spent;
b) regenerating a spent primary oxygenate adsorption unit via a first regenerant stream to provide an oxygenated first regenerant stream comprising the water and the oxygenates;
c) removing a portion of the water from the oxygenated first regenerant stream;
d) removing a residual water and the oxygenates from the oxygenated first regenerant stream via a secondary oxygenate adsorption unit to provide a deoxygenated first regenerant stream, wherein the secondary oxygenate adsorption unit becomes spent; and
e) regenerating a spent secondary oxygenate adsorption unit via a second regenerant stream to provide an oxygenated second regenerant stream comprising the residual water and the oxygenates.

2. The process of claim 1, further comprising:
f) permanently removing the oxygenated second regenerant stream from the light hydrocarbon processing system.

3. The process of claim 1, further comprising:
g) prior to step b), recovering residual olefins from the spent primary oxygenate adsorption unit.

4. The process of claim 3, wherein step g) comprises flushing the residual olefins from the spent primary oxygenate adsorption unit with isobutane.

5. The process of claim 1, wherein step c) comprises: cooling the oxygenated first regenerant stream, and condensing the portion of the water from the oxygenated first regenerant stream.

6. The process of claim 1, wherein the first regenerant stream has a temperature of at least 121.1 degree Celsius (250 degree Fahrenheit).

7. The process of claim 1, wherein the second regenerant stream comprises fuel gas.

8. The process of claim 7, wherein step f) comprises combusting the oxygenated second regenerant stream.

9. The process of claim 1, wherein:
the olefin stream fed to the primary oxygenate adsorption unit has a water content of at least 300 ppmw, and
the deoxygenated olefin stream provided by the primary oxygenate adsorption unit has an oxygenate content of not more than 5 ppmw and the water content of not more than 5 ppmw.

10. The process of claim 1, further comprising:
h) contacting the deoxygenated olefin stream and an isoparaffin stream with an ionic liquid catalyst in an ionic liquid alkylation zone under ionic liquid alkylation conditions to provide an ionic liquid alkylate.

11. A process for eliminating oxygenates from a light hydrocarbon processing system, the process comprising:
a) adsorbing water and the oxygenates from an olefin stream via a primary oxygenate adsorption unit to provide a deoxygenated olefin stream;
b) when the primary oxygenate adsorption unit is spent, desorbing the water and the oxygenates from the primary oxygenate adsorption unit via a first regenerant stream to provide an oxygenated first regenerant stream comprising the water and the oxygenates;
c) removing a portion of the water from the oxygenated first regenerant stream as condensate;
d) via a secondary oxygenate adsorption unit, adsorbing residual water and the oxygenates from the oxygenated first regenerant stream;
e) via a second regenerant stream, desorbing a residual water and the oxygenates from a secondary oxygenate adsorption unit to provide an oxygenated second regenerant stream comprising the residual water and the oxygenates; and
f) permanently removing the oxygenated second regenerant stream from the light hydrocarbon processing system.

12. The process of claim 11, wherein the second regenerant stream comprises fuel gas.

13. The process of claim 11, further comprising:
g) combusting the oxygenated second regenerant stream.

14. The process of claim 11, further comprising:
h) permanently removing a condensate water from the light hydrocarbon processing system.

15. The process of claim 11, further comprising:
i) after step a), contacting the deoxygenated olefin stream and at least one isoparaffin with an ionic liquid catalyst in an ionic liquid alkylation zone under ionic liquid alkylation conditions;
j) separating an alkylation hydrocarbon phase from an effluent of the ionic liquid alkylation zone; and
k) fractionating the alkylation hydrocarbon phase to provide an alkylate product.

16. A process for eliminating oxygenates from a light hydrocarbon processing system, the process comprising:
a) feeding an olefin stream to a primary oxygenate adsorption unit to provide a deoxygenated olefin stream;
b) contacting the deoxygenated olefin stream and an isoparaffin stream with an ionic liquid catalyst in an ionic liquid alkylation zone under ionic liquid alkylation conditions;
c) separating an alkylation hydrocarbon phase from an effluent of the ionic liquid alkylation zone;
d) fractionating the alkylation hydrocarbon phase to provide an alkylate product;
e) when the primary oxygenate adsorption unit becomes spent, regenerating a spent primary oxygenate adsorption unit via a first regenerant stream to provide an oxygenated first regenerant stream;
f) removing the oxygenates from the oxygenated first regenerant stream via a secondary oxygenate adsorption unit to provide a deoxygenated first regenerant stream; and
g) when the secondary oxygenate adsorption unit becomes spent, regenerating a spent secondary oxygenate adsorption unit via a second regenerant stream to provide an oxygenated second regenerant stream.

17. The process of claim 16, wherein the oxygenated first regenerant stream comprises water, and the process further comprises:
h) prior to step f), removing a portion of the water from the oxygenated first regenerant stream as condensate.

18. The process of claim 16, wherein:
step a) comprises removing water and the oxygenates from the olefin stream via the primary oxygenate adsorption unit, and
the deoxygenated olefin stream has a water content of not more than 5 ppmw and an oxygenate content of not more than 5 ppmw.

19. The process of claim 16, wherein:
the isoparaffin stream comprises the deoxygenated first regenerant stream, and
the deoxygenated first regenerant stream has a water content of not more than 50 ppmw and an oxygenate content of not more than 5 ppmw.

20. The process of claim 16, further comprising:
i) combusting the oxygenated second regenerant stream.
